(19) United States
(12) Patent Application Publication
(10) Pub. No.: US 2023/0302032 A1
(43) Pub. Date: Sep. 28, 2023

(54) ANTIBACTERIAL COMPOSITIONS AND METHODS FOR FABRICATING THEREOF

(71) Applicant: Harrow IP LLC, Nashville, TN (US)

(72) Inventors: Dennis Elias Saadeh, Nashville, TN (US); Mark L. Baum, Nashville, TN (US)

(21) Appl. No.: 18/019,580

(22) PCT Filed: Aug. 4, 2021
(86) PCT No.: PCT/US2021/044518

§ 371 Date: Feb. 3, 2023

Related U.S. Application Data
(60) Provisional application No. 63/060,829, filed on Aug. 4, 2020.

(51) Int. Cl. A61K 31/7048; A61K 31/167; A61K 31/7036; A61K 31/198

(52) U.S. Cl. CPC A61K 31/7048; A61K 31/167; A61K 31/7036; A61K 31/198

(57) ABSTRACT

Pharmaceutical compositions are described, the compositions comprising a therapeutically effective quantity of at least one glycopeptide antibiotic, at least one aminoglycoside antibiotic, and at least one amino acid. Methods for fabricating the compositions and using them are also described.

### CROSS-REFERENCE TO RELATED APPLICATIONS

This application claims priority to U.S. Provisional Application No. 63/060,829 filed Aug. 4, 2020, the entirety of which is incorporated by reference herein.

### FIELD OF THE INVENTION

The present disclosure relates generally to the field of ophthalmology and more specifically to pharmaceutical compositions for the treatment of bacterial eye infections such as microbial keratitis.

### BACKGROUND

Bacterial infections that frequently arise in connection with many diseases and disorders present a serious problem, especially in the case of antibiotic resistant bacteria. This problem may be particularly acute in case of bacterial eye infections, which account for about 90% of cases of microbial keratitis characterized by stromal loss with an overlying epithelial defect. Typically, the bacteria that are responsible for such infections are staphylococci and pseudomonades.

A variety of antibiotics are in use for treating microbial keratitis. Such antibiotics as vancomycin, tobramycin, cefazolin, fluroquinolones, and combinations of these are frequently used. However, using such antibiotics is not free of several drawbacks and deficiencies. For example, Staphylococcus aureus bacteria are known to develop resistance to many antibiotics such as ciprofloxacin, moxifloxacin, tobramycin, gentamycin, azithromycin, etc., but usually not vancomycin. Using vancomycin, however, has its own problems and drawbacks.

More specifically, vancomycin is not commercially available and must be compounded, and delays in obtaining the antibiotic due to the need for compounding can have a negative effect on the symptom resolution and treatment. Also, compounded vancomycin is not typically tested for potency or sterility due to the need for immediate therapy and must be stored frozen with a short shelf life even under frozen conditions. In addition, compounded vancomycin is not ready-to-use and presents compliance issues that could have harmful sequelae.

In view of the foregoing, it is therefore desirable to have alternative compounded antibiotics that are safer but equally effective. This disclosure provides such alternatives and describes stabilized formulations of vancomycin that can not only be stored under refrigerated conditions but at room temperature once opened for patient use and is preserved for multiple dose application and portability. The described compositions do not require reconstitution or thawing, may be dispensed in traditional multi-dose eye dropper bottles for ease of administration, and are ready to stock in hospitals, emergency rooms, and clinics where immediately needed to treat infections. Finally, the described compositions reduce time and costs of compounding, reduce the possibility of contamination, and exhibits the same antibacterial activity as commercial vancomycin.

### SUMMARY

Described herein is an ophthalmic pharmaceutical composition comprising a therapeutically effective quantity of at least one of a glycopeptide antibiotic and an aminoglycoside antibiotic, at least one amino acid, and a pharmaceutically acceptable aqueous carrier therefor. The ophthalmic pharmaceutical composition may have a pH of between about 4.0 and about 6.0. The pharmaceutical ophthalmic composition may be stable for at least about 30 days up to at least about 365 days when refrigerated. The pharmaceutical ophthalmic composition may be potent for at least about 30 days up to at least about 365 days when refrigerated.

The glycopeptide antibiotic may include vancomycin, decaplanin, ramoplanin, cefuroxime, teicoplanin, telavancin, corbomycin, complestatin, and any combination thereof. The glycopeptide antibiotic may have a concentration in the ophthalmic pharmaceutical composition of between about 1 mass % to about 10 mass %.

The aminoglycoside antibiotic may include tobramycin, kanamycin A, amikacin, dibekacin, gentamicin, sisomicin, netilmicin, neomycin B, neomycin C, paromomycin, plazomicin, streptomycin, and any combination thereof. The aminoglycoside antibiotic may have a concentration in the ophthalmic pharmaceutical composition of between about 1 mass % to about 5 mass %.

The at least one amino acid may include L-glycine, L-cysteine, L-lysine, N-acetyl-D-alanine, and any combination thereof. The at least one amino acid may have a concentration in the ophthalmic pharmaceutical composition of between about 0.1 mass % to about 1.5 mass %.

The ophthalmic pharmaceutical composition may further comprise at least one anesthetic. The at least one anesthetic may include lidocaine, tetracaine, proparacaine, procaine, dyclonine, chloroprocaine, and any combination thereof.

Further described herein is a method for treating an ophthalmological disease, condition, disorder, syndrome or pathology in a mammalian subject in need of such treatment, the method comprising administering to a patient in need thereof a pharmaceutically effective quantity of a composition described herein. The ophthalmological disease, condition, disorder, syndrome, or pathology may include microbial keratitis, endophthalmitis prophylaxis post cataract surgery, and infection prophylaxis for corneal prosthetic implant. The ophthalmic pharmaceutical composition may be administered topically. The ophthalmic pharmaceutical composition may be administered in the form of eyedrops.

### DETAILED DESCRIPTION

### A. Terms, Definitions and Abbreviations

It is to be understood that this disclosure is not limited to the particular methods, compositions, or materials specified herein, but is extended to equivalents thereof as would be recognized by those ordinarily skilled in the relevant arts. It should also be understood that terminology employed herein is used for the purpose of describing particular embodiments only and is not intended to be limiting.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL” The endpoint may also be based on the variability allowed by an appropriate regulatory body, such as the FDA, USP, etc.

As used herein, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

The term “pharmaceutical composition” is defined as a chemical or biological compound or substance, or a mixture or combination of two or more such compounds or substances, intended for use in the medical diagnosis, cure, treatment, or prevention of a disease or pathology.

The term “glycopeptide antibiotic” refers to a type of antibiotic that inhibits bacterial cell wall formation by inhibiting peptidoglycan synthesis. Glycopeptide antibiotics include glycosylated cyclic or polycyclic nonribosomal peptide moieties.

The term “vancomycin” refers to a specific glycopeptide antibiotic having the IUPAC name (1S,2R,18R,19R,22S,25R,28R,40S)-48-{[(2S,3R,4S,5S,6R)-3-{[(2S,4S,5S,6S)-4-amino-5-hydroxy-4,6-dimethyloxan-2-yl]oxy}-4,5-dihydroxy-6-(hydroxymethyl)oxan-2-yl]oxy}-22-(carbamoylmethyl)-5,15-dichloro-2,18,32,35,37-pentahydroxy-19-[(2R)-4-methyl-2-(methyl amino)pentanamido]-20,23,26,42,44-pentaoxo-7,13-dioxa-21,24,27,41,43-pentaazaoctacyclo [26.14.2.23,6.214,17.18,12.129,33.010,25.034,39]-pentaconta-5,8(48),9,11,14,16,29(45),30,32,34,36,38,46,49-pentadecaene-40-carboxylic acid and the following chemical structure:

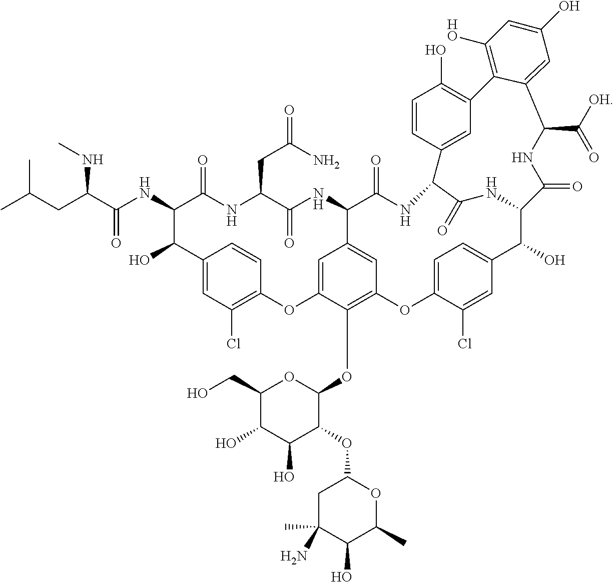

Vancomycin is available under the trade name VANCOCIN® from Eli Lilly & Co. of Indianapolis, Indiana, and from other suppliers.

The term “aminoglycoside antibiotic” refers to a type of antibiotic that inhibits protein synthesis and contains as a portion of the molecule an amino-modified glycoside.

The term “tobramycin” refers to a specific aminoglycoside antibiotic having the IUPAC name (2S,3R,4S,5S,6R)-4-amino-2-{[(1S,2S,3R,4S,6R)-4,6-diamino-3-{[(2R,3R,5S,6R)-3-amino-6-(aminomethyl)-5-hydroxyoxan-2-yl]oxy}-2-hydroxycyclohexyl]oxy}-6-(hydroxymethyl)oxane-3,5-diol and the following chemical structure:

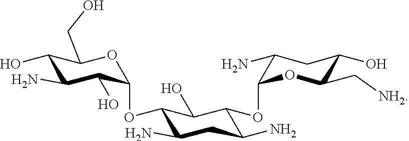

Tobramycin is available under the trade name TOBREX® from Bausch & Lomb Pharmaceuticals of Bridgewater, New Jersey, and from other suppliers.

The term “amino acid” refers herein to amino acids known to those having skill in the art as well as derivatives of amino acids including, but not limited to, acetylated derivatives of amino acids.

The term “anesthetic” refers herein to a substance or compound that induces temporary insensitivity to pain such as a temporary loss of sensation.

The term “lidocaine” refers to 2-diethylamino-N-(2,6-dimethylphenyl)acetamide, a chemical compound having the following chemical structure:

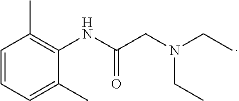 [SMILES: CCN(CC)CC(=O)Nc1c(C)cccc1C]

Lidocaine is available under the trade name XYLOCAINE® from a variety of suppliers.

The term “carrier” refers to a substance that serves as a vehicle for improving the efficiency of delivery and the effectiveness of a pharmaceutical composition. One non-limiting example of a carrier for the purposes of the instant application is water such as de-ionized sterile water.

The term “microbial keratitis” refers to an infection of the cornea that is caused by bacteria.

As used herein, “therapeutically effective quantity” refers to an amount that leads to measurable and beneficial effects for the subject administered the substance.

As used herein, the terms “treat,” “treating,” or “treatment” refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilized (i.e. not worsening) state of disease, a delay or slowing of disease progression, amelioration or palliation of the disease state, and remission (whether partial or total), whether detectable or undetectable. In the context of the present disclosure, beneficial or desired clinical results may include improvement in visual acuity as measured by a Jaeger eye test. “Treatment” can also mean prolonging survival as compared to expected survival if not receiving treatment. Those in need of treatment include those already with the disease, condition, or disorder as well as those prone to have the disease, condition or disorder or those in which the disease, condition, or disorder is to be prevented.

The term “pharmaceutically acceptable” when used to define a carrier, whether diluent or excipient, or another substance to be included in the compositions described herein, refers to a substance that is compatible with the other ingredients of the formulation and not deleterious to the recipient thereof.

The terms “administration of a composition” or “administering a composition” are defined to include the act of providing a compound or pharmaceutical composition of the application to the subject in need of treatment.

The term “stability” refers to the slow or lacking degradation of one or more active ingredients of a pharmaceutical composition. Stated another way, a stable composition is one in which one or more active ingredients have not degraded or decomposed over a period of time and the concentration of one or more active ingredients has not significantly changed over a period of time.

The term “potent” or “potency” refers to the concentration of one or more active ingredients in a pharmaceutical composition in reference to the amount of the one or more active ingredients needed to produce a therapeutic effect. As a non-limiting example, a hypothetical pharmaceutical composition may require 1% (w/v) of an active ingredient to have a therapeutic effect. The hypothetical composition would lack potency if it only contained 0.1% (w/v) of the active ingredient.

### B. Compositions

Disclosed herein are compositions comprising a therapeutically effective quantity of at least one antibiotic selected from the group consisting of a glycopeptide antibiotic and an aminoglycoside antibiotic, and at least one amino acid.

### (a) Antibiotic

The compositions of the present disclosure includes a therapeutically effective quantity of at least one antibiotic selected from the group consisting of a glycopeptide antibiotic and an aminoglycoside antibiotic. In some aspects, the composition may include a glycopeptide antibiotic, an aminoglycoside antibiotic, or both.

### (i) Glycopeptide Antibiotic

In some embodiments, the antibiotic may include a glycopeptide antibiotic or pharmaceutically acceptable salts thereof. The glycopeptide antibiotic may include vancomycin, decaplanin, ramoplanin, cefuroxime, teicoplanin, telavancin, corbomycin, complestatin, other glycopeptide antibiotics known in the art, and combinations thereof.

In some embodiments, the glycopeptide antibiotic may have a concentration in the pharmaceutical composition of the present disclosure of between about 1.0 mass % and about 10.0 mass %. In some aspects, the glycopeptide antibiotic may have a concentration of between about 1.0 mass % to about 2.0 mass %, about 2.0 mass % to about 3.0 mass %, about 3.0 mass % to about 4.0 mass %, about 4.0 mass % to about 5.0 mass %, about 5.0 mass % to about 6.0 mass %, about 6.0 mass % to about 7.0 mass %, about 7.0 mass % to about 8.0 mass %, about 8.0 mass % to about 9.0 mass %, or about 9.0 mass % to about 10.0 mass %. In some additional aspects, the glycopeptide antibiotic may have a concentration in the pharmaceutical composition between about 2.0 mass % to about 9.0 mass %, about 3.0 mass % to about 8.0 mass %, or about 4.0 mass % to about 6.0 mass %. In still further aspects, the glycopeptide antibiotic may have a concentration of about 1.0 mass %, 2.0 mass %, 3.0 mass %, 4.0 mass %, 5.0 mass %, 6.0 mass %, 7.0 mass %, 8.0 mass %, 9.0 mass %, or about 10.0 mass %. In one example, the glycopeptide antibiotic is vancomycin and has a concentration in the pharmaceutical composition of about 0.46 mass %.

### (ii) Aminoglycoside Antibiotic

In some embodiments, the antibiotic may include an aminoglycoside antibiotic or pharmaceutically acceptable salts thereof. The aminoglycoside antibiotic may include tobramycin, kanamycin A, amikacin, dibekacin, gentamicin, sisomicin, netilmicin, neomycin B, neomycin C, paromomycin, plazomicin, streptomycin, or other aminoglycoside antibiotics known in the art and any combination thereof.

In some embodiments, the aminoglycoside antibiotic may have a concentration in the pharmaceutical composition between about 1.0 mass % to about 5.0 mass %. In some aspects, the aminoglycoside antibiotic may have a concentration in the pharmaceutical composition of between about 1.0 mass % to about 2.0 mass %, about 2.0 mass % to about 3.0 mass %, about 3.0 mass % to about 4.0 mass %, or about 4.0 mass % to about 5.0 mass %. In some additional aspects, the concentration of the aminoglycoside antibiotic may be about 1.0 mass %, 1.5 mass %, 2.0 mass %, 2.5 mass %, 3.0 mass %, 3.5 mass %, 4.0 mass %, 4.5 mass %, or about 5.0 mass %. In one example, the aminoglycoside antibiotic is tobramycin HCl with a concentration of about 1.5 mass %.

### (b) Amino Acid

The composition of the present disclosure includes at least one amino acid. Without being bound by theory, the at least one amino acid may be useful to stabilize the antibiotic(s) to allow for longer storage of the composition. Alternatively, or additionally, the at least one amino acid may act as a tonicity adjusting agent. In some embodiments, the amino acid may be at least one of L-glycine, L-cysteine, L-lysine, N-acetyl-D-alanine, or any other amino acid or combinations thereof. In one non-limiting example, the at least one amino acid includes a combination of L-lysine and N-acetyl-D-alanine.

The at least one amino acid may have a concentration in the composition of between about 0.1 mass % to about 1.5 mass %. In some embodiments, the at least one amino acid may have a concentration of between about 0.3 mass % to about 1.2 mass %, or between about 0.5 mass % to about 1.0 mass %. In some additional embodiments, the at least one amino acid may have a concentration of between about 0.1 mass % to about 0.2 mass %, about 0.2 mass % to about 0.3 mass %, about 0.3 mass % to about 0.4 mass %, about 0.4 mass % to about 0.5 mass %, about 0.5 mass % to about 0.6 mass %, about 0.6 mass % to about 0.7 mass %, about 0.7 mass % to about 0.8 mass %, about 0.8 mass % to about 0.9 mass %, about 0.9 mass % to about 1.0 mass %, about 1.0 mass % to about 1.1 mass %, about 1.1 mass % to about 1.2 mass %, about 1.2 mass % to about 1.3 mass %, about 1.3 mass % to about 1.4 mass %, or about 1.4 mass % to about 1.5 mass %. In yet additional embodiments, the at least one amino acid may have a concentration in the composition of about 0.1 mass %, 0.2 mass %, 0.3 mass %, 0.4 mass %, 0.5 mass %, 0.6 mass %, 0.7 mass %, 0.8 mass %, 0.9 mass %, 1.0 mass %, 1.1 mass %, 1.2 mass %, 1.3 mass %, 1.4 mass %, or about 1.5 mass %.

In some embodiments, the amino acid includes L-glycine. In some aspects, the L-glycine may have a concentration in the composition of between about 0.1 mass % to about 1.0 mass %. In some additional aspects, the L-glycine may have a concentration in the composition of between about 0.2 mass % to about 0.95 mass %, about 0.4 mass % to about 0.9 mass %, or about 0.6 mass % to about 0.85 mass %. In yet other aspects, the L-glycine may have a concentration in the composition of between about 0.1 mass % to about 0.2 mass %, about 0.2 mass % to about 0.3 mass %, about 0.3 mass % to about 0.4 mass %, about 0.4 mass % to about 0.5 mass %, about 0.5 mass % to about 0.6 mass %, about 0.6 mass % to about 0.7 mass %, about 0.7 mass % to about 0.8 mass %, about 0.8 mass % to about 0.9 mass %, or about 0.9 mass % to about 1.0 mass %. In still further aspects, the L-glycine may have a concentration in the composition of about 0.1 mass %, 0.15 mass %, 0.2 mass %, 0.25 mass %, 0.3 mass %, 0.35 mass %, 0.4 mass %, 0.45 mass %, 0.5 mass %, 0.55 mass %, 0.6 mass %, 0.65 mass %, 0.7 mass %, 0.75 mass %, 0.8 mass %, 0.85 mass %, 0.9 mass %, 0.95 mass %, or about 1.0 mass %. In one example, the L-glycine has a concentration of about 0.83 mass %.

In some other embodiments, the amino acid includes L-cysteine. In some aspects, the L-cysteine may have a concentration in the composition between about 0.1 mass % and about 0.2 mass %. In some additional aspects, the concentration of L-cysteine in the composition may be between about 0.1 mass % to about 0.125 mass %, about 0.125 mass % to about 0.15 mass %, about 0.15 mass% to about 0.175 mass %, or about 0.175 mass % to about 0.2 mass %. In yet additional aspects, the L-cysteine may have a concentration in the composition of about 0.1 mass %, 0.11 mass %, 0.12 mass %, 0.13 mass %, 0.14 mass %, 0.15 mass %, 0.16 mass %, 0.17 mass %, 0.18 mass %, 0.19 mass %, or about 0.2 mass %. In some examples, the L-cysteine may have a concentration in the composition of about 0.2 mass %.

In yet other embodiments, the amino acid includes L-lysine. In some aspects, the L-lysine has a concentration in the composition of between about 0.1 mass % to about 1.0 mass %. In some additional aspects, the L-lysine has a concentration in the composition of between about 0.1 mass % to about 0.2 mass %, about 0.2 mass % to about 0.3 mass %, about 0.3 mass % to about 0.4 mass %, about 0.4 mass % to about 0.5 mass %, about 0.5 mass % to about 0.6 mass %, about 0.6 mass % to about 0.7 mass %, about 0.7 mass % to about 0.8 mass %, about 0.8 mass % to about 0.9 mass %, or about 0.9 mass % to about 1.0 mass %. In yet additional aspects, the L-lysine has a concentration in the composition of about 0.1 mass %, 0.2 mass %, 0.3 mass %, 0.4 mass %, 0.5 mass %, 0.6 mass %, 0.7 mass %, 0.8 mass %, 0.9 mass%, or about 1.0 mass %. In some examples, the L-lysine has a concentration in the composition of about 0.9 mass %.

In still other embodiments, the amino acid includes N-acetyl-D-alanine. In some aspects, the N-acetyl-D-alanine has a concentration in the composition of between about 0.25 mass % to about 1.5 mass %. In some additional aspects, the N-acetyl-D-alanine has a concentration in the composition of between about 0.3 mass% to about 1.2 mass %, about 0.4 mass % to about 0.8 mass %, or about 0.5 mass % to about 0.6 mass %. In yet additional aspects, the N-acetyl-D-alanine has a concentration in the composition of between about 0.25 mass % to about 0.3 mass %, about 0.3 mass % to about 0.4 mass %, about 0.4 mass % to about 0.5 mass %, about 0.5 mass % to about 0.6 mass %, about 0.6 mass % to about 0.7 mass %, about 0.7 mass % to about 0.8 mass %, about 0.8 mass % to about 0.9 mass %, about 0.9 mass % to about 1.0 mass %, about 1.0 mass % to about 1.1 mass %, about 1.1 mass % to about 1.2 mass %, about 1.2 mass % to about 1.3 mass %, about 1.3 mass % to about 1.4 mass %, or about 1.4 mass % to about 1.5 mass %. In still additional aspects, the N-acetyl-D-alanine may have a concentration in the composition of about 0.25 mass %, 0.3 mass %, 0.4 mass %, 0.5 mass %, 0.6 mass %, 0.7 mass %, 0.8 mass %, 0.9 mass %, 1.0 mass %, 1.1 mass %, 1.2 mass %, 1.3 mass %, 1.4 mass %, or about 1.5 mass %. In some examples, the N-acetyl-D-alanine has a concentration in the composition of about 0.5 mass %.

### (c) Anesthetic

In some embodiments, the composition of the present disclosure may include an anesthetic. The anesthetic may include lidocaine, tetracaine, proparacaine, procaine, dyclonine, chloroprocaine, pharmaceutically acceptable salts thereof, and any combination thereof. Without being bound by theory, the anesthetic may impart greater stability to the pharmaceutical composition. In some examples, the anesthetic is lidocaine. In some other examples, the anesthetic is lidocaine HCl.

In some embodiments, the anesthetic may have a concentration in the composition of the present disclosure of between about 0.25 mass % to about 5.0 mass %. In some aspects, the anesthetic may have a concentration in the composition of the present disclosure of between about 1.0 mass % to about 4.0 mass % or about 2 mass % to about 3 mass %. In some additional aspects, the anesthetic may have a concentration in the composition of between about 0.25 mass % to about 0.5 mass %, about 0.5 mass % to about 1.0 mass %, about 1.0 mass % to about 1.5 mass %, about 1.5 mass % to about 2.0 mass %, about 2.0 mass % to about 2.5 mass %, about 2.5 mass % to about 3.0 mass %, about 3.0 mass % to about 3.5 mass %, about 3.5 mass % to about 4.0 mass %, about 4.0 mass % to about 4.5 mass %, or about 4.5 mass % to about 5.0 mass %. In yet additional aspects, the anesthetic may have a concentration in the composition of about 0.25 mass %, 0.5 mass %, 1.0 mass %, 1.5 mass %, 2.0 mass %, 2.5 mass %, 3.0 mass %, 3.5 mass %, 4.0 mass %, 4.5 mass %, or about 5.0 mass %. In one example, the anesthetic is lidocaine having a concentration of about 1.0 mass %.

### (d) Exemplary Embodiments

In one exemplary embodiment, the composition of the present disclosure comprises a glycopeptide antibiotic, an aminoglycoside antibiotic, and an amino acid, wherein the glycopeptide antibiotic is vancomycin having a concentration of about 4.6 mass % in the composition, the aminoglycoside antibiotic is tobramycin HCl having a concentration of about 1.6 mass % in the composition, and the amino acid is L-glycine having a concentration of about 0.83 mass % in the composition.

### (e) Pharmaceutical Formulations

In some embodiments, compositions of the present disclosure are pharmaceutically-acceptable formulations. These pharmaceutically-acceptable formulations may be used to treat, prevent, or alleviate bacterial keratitis. In some aspects, the pharmaceutically-acceptable formulations may be sterile. The pharmaceutically-acceptable formulations may include pharmaceutically-acceptable excipients, including solvents, pH adjusting agents, buffering agents, antioxidants, tonicity modifying agents, osmotic adjusting agents, preservatives, stabilizing agents, viscosity adjusting agents, surfactants, or any other pharmaceutically-acceptable excipients known in the art or combinations thereof. In some examples of the present disclosure, the pharmaceutically-acceptable formulations include benzalkonium chloride.

The pharmaceutically-acceptable formulations may also include a pharmaceutically-acceptable carrier. A pharmaceutically-acceptable carrier may be any substance that serves as a vehicle for improving the efficiency of delivery and the effectiveness of the pharmaceutical composition. In some examples of the present disclosure, the pharmaceutically-acceptable carrier may include water (e.g., deionized water or sterile water for injection). In some examples of the present disclosure, the pharmaceutically-acceptable carrier may include hypromellose.

In some embodiments, the pharmaceutically-acceptable formulation may have a pH of between about 4.0 to about 6.0. In some aspects, pharmaceutically-acceptable formulation may have a pH of between about 4.0 to about 4.5, about 4.5 to about 5.0, about 5.0 to about 5.5, or about 5.5 to about 6.0. In some additional aspects, the pharmaceutically acceptable formulation may have a pH of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or about 6.0. In some examples, the pharmaceutically-acceptable formulation may have a pH of between about 5.1 to about 5.3.

In some embodiments, the pharmaceutically-acceptable formulation may be stable for at least about 30 days when refrigerated. In some aspects, the pharmaceutically-acceptable formulation may be stable for at least about 30, 60, 90, 120, 150, 180, 210, 240, 270, 300, 330, or about 365 days when refrigerated. In some examples, the pharmaceutically-acceptable formulation may be stable for about 120 days to about 150 days when refrigerated.

In some embodiments, the pharmaceutically-acceptable formulation may be potent for at least about 30 days when refrigerated. In some aspects, the pharmaceutically-acceptable formulation may be potent for at least about 30, 60, 90, 120, 150, 180, 210, 240, 270, 300, 330, or about 365 days when refrigerated.

In some aspects, the glycopeptide antibiotic in the pharmaceutically-acceptable formulation may have a concentration of at least 100% compared to the initial concentration of the antibiotic after at least about 30 days when refrigerated; stated another way, the concentration of the glycopeptide antibiotic on day 30 may be at least 100% of the concentration of the antibiotic on day zero. In some additional aspects, the glycopeptide antibiotic in the pharmaceutically-acceptable formulation may have a concentration of at least 100% compared to the initial concentration of the antibiotic after at least about 30, 60, 90, 120, 150, 180, 210, 240, 270, 300, 330, or about 365 days when refrigerated. In some examples, the glycopeptide antibiotic in the pharmaceutically-acceptable formulation may have a concentration of at least 100% compared to the initial concentration of the antibiotic after about 180 days when refrigerated.

In some aspects, the aminoglycoside antibiotic in the pharmaceutically-acceptable formulation may have a concentration of at least 100% compared to the initial concentration of the antibiotic after at least about 30 days when refrigerated; stated another way, the concentration of the aminoglycoside antibiotic on day 30 may be at least 100% of the concentration of the antibiotic on day zero. In some additional aspects, the glycopeptide antibiotic in the pharmaceutically-acceptable formulation may have a concentration of at least 100% compared to the initial concentration of the antibiotic after at least about 30, 60, 90, 120, 150, 180, 210, 240, 270, 300, 330, or about 365 days when refrigerated. In some examples, the aminoglycoside antibiotic in the pharmaceutically-acceptable formulation may have a concentration of at least 100% compared to the initial concentration of the antibiotic after about 180 days when refrigerated.

### C. Methods

Disclosed herein are methods of making and using a composition of the present disclosure.

a. Method of Making

Further disclosed herein are methods of making a pharmaceutical formulation comprising a therapeutically effective quantity of at least one antibiotic selected from the group consisting of a glycopeptide antibiotic and an aminoglycoside antibiotic and at least one amino acid. The composition made by the disclosed method may be any composition described in Section B above.

In some embodiments, the method is a single-batch method, which includes combining all of the components of the composition in a single container. In some aspects, the components may be added to the container simultaneously or consecutively. In other embodiments, the method may be a two- or multiple-batch method, which may include combining each component of the disclosed composition in separate containers followed by combining the contents of each container.

b. Methods of Treatment

Further disclosed herein are methods of treating a bacterial infection that is concomitant with an ophthalmological disease, condition, disorder, syndrome, or pathology in a mammalian subject, by administering a therapeutically effective amount of a composition of the present disclosure to the subject. The composition may be any of the compositions described in Section B. It will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, gender, diet of the patient being treated, and the severity of the particular ophthalmological condition being treated.

In some embodiments, the ophthalmological disease, condition, disorder, syndrome, or pathology to be treated may include infections resulting from cataract surgery, glaucoma surgery, surgery to treat diseases of the retina, etc. In some aspects, the ophthalmological disease, condition, disorder, syndrome, or pathology may include microbial keratitis, endophthalmitis, prophylaxis post-cataract surgery, and infection prophylaxis for corneal prosthetic implant. In some examples, the ophthalmological disease, condition, disorder, syndrome, or pathology may include microorganism-naïve bacterial keratitis; i.e., keratitis caused by an unknown bacterial pathogen.

In some embodiments, the composition may be administered topically through eyedrops. In some embodiments, the eyedrops may be administered at least once every hour, once every two hours, once every three hours, once every four hours, once every five hours, once every six hours, and so on. In some additional embodiments the eyedrops may be administered only during waking hours. In still other embodiments, the eyedrops may be administered for a period of between about 3 days to about 14 days, wherein the dosage rate may be varied over the course of treatment. In one example, the eyedrops are administered at least once every waking hour for at least 72 hours, and then three to four times per day for at least 7 days.

### D. Kits

In some embodiments, pharmaceutical kits are provided that contain a composition of the present disclosure. The kit may include a sealed container approved for the storage of pharmaceutical compositions wherein the container contains one of the above-described pharmaceutical compositions. The kit may further include an instruction for the use of the composition and information about the composition.

### EXEMPLARY EMBODIMENTS

Described herein are exemplary embodiments of the present disclosure.

Embodiment 1: An ophthalmic pharmaceutical composition, comprising:
(a) a therapeutically effective quantity of at least one antibiotic selected from the group consisting of a glycopeptide antibiotic and an aminoglycoside antibiotic;
(b) at least one amino acid; and
(c)a pharmaceutically acceptable aqueous carrier therefor.

Embodiment 2: The ophthalmic pharmaceutical composition of embodiment 1, wherein the glycopeptide antibiotic is selected from the group consisting of vancomycin, decaplanin, ramoplanin, cefuroxime, teicoplanin, telavancin, corbomycin, complestatin, and any combination thereof.

Embodiment 3: The ophthalmic pharmaceutical composition of embodiment 2, wherein the glycopeptide antibiotic is vancomycin.

Embodiment 4: The ophthalmic pharmaceutical composition of embodiment 1, wherein the aminoglycoside antibiotic is selected from the group consisting of tobramycin, kanamycin A, amikacin, dibekacin, gentamicin, sisomicin, netilmicin, neomycin B, neomycin C, paromomycin, plazomicin, streptomycin, and any combination thereof.

Embodiment 5: The ophthalmic pharmaceutical composition of embodiment 4, wherein the aminoglycoside antibiotic is tobramycin.

Embodiment 6: The ophthalmic pharmaceutical composition of embodiment 1, further comprising a therapeutically effective quantity of at least one anesthetic compound.

Embodiment 7: The ophthalmic pharmaceutical composition of embodiment 6, wherein the at least one anesthetic compound is selected from the group consisting of lidocaine, tetracaine, proparacaine, procaine, dyclonine, chloroprocaine, and any combination thereof.

Embodiment 8: The ophthalmic pharmaceutical composition of embodiment 6, wherein the at least one anesthetic compound has a concentration in the ophthalmic pharmaceutical composition of between about 0.25 mass % and about 5.0 mass %.

Embodiment 9: The ophthalmic pharmaceutical composition of embodiment 8, wherein the at least one anesthetic compound has a concentration of about 1.0 mass %.

Embodiment 10: The ophthalmic pharmaceutical composition of embodiment 7, wherein the anesthetic compound is lidocaine.

Embodiment 11: The ophthalmic pharmaceutical composition of embodiment 1, wherein the concentration of glycopeptide antibiotic(s) is between about 1.0 mass % and about 10.0 mass %.

Embodiment 12: The ophthalmic pharmaceutical composition of embodiment 1, wherein the concentration of aminoglycoside antibiotic(s) is between about 1.0 mass % and about 5.0 mass %.

Embodiment 13: The ophthalmic pharmaceutical composition of embodiment 1, wherein the composition has a pH of between about 4.0 and about 6.0.

Embodiment 14: The ophthalmic pharmaceutical composition of embodiment 13, wherein the composition has a pH of between about 5.1 to about 5.3.

Embodiment 15: The ophthalmic pharmaceutical composition of embodiment 1, wherein the at least one amino acid is selected from the group consisting of L-glycine, L-cysteine, L-lysine, N-acetyl-D-alanine, and any combination thereof.

Embodiment 16: The ophthalmic pharmaceutical composition of embodiment 14, wherein the at least one amino acid has a concentration in the pharmaceutical composition between about 0.1 mass % and about 1.5 mass %.

Embodiment 17: The ophthalmic pharmaceutical composition of embodiment 16, wherein the at least one amino acid is L-glycine and the L-glycine has a concentration in the ophthalmic pharmaceutical composition of between about 0.1 mass % to about 1.0 mass %.

Embodiment 18: The ophthalmic pharmaceutical composition of embodiment 17, wherein the L-glycine has a concentration of about 0.8 mass %.

Embodiment 19: The ophthalmic pharmaceutical composition of embodiment 16, wherein the at least one amino acid is L-cysteine and the L-cysteine has a concentration in the ophthalmic pharmaceutical composition of between about 0.1 mass % and about 0.2 mass %.

Embodiment 20: The ophthalmic pharmaceutical composition of embodiment 19, wherein the L-cysteine has a concentration of about 0.2 mass %.

Embodiment 21: The ophthalmic pharmaceutical composition of embodiment 16, wherein the at least one amino acid is L-lysine and the L-lysine has a concentration in the ophthalmic pharmaceutical composition of between about 0.1 mass % to about 1.0 mass %.

Embodiment 22: The ophthalmic pharmaceutical composition of embodiment 21, wherein the L-lysine has a concentration of about 0.9 mass %.

Embodiment 23: The ophthalmic pharmaceutical composition of embodiment 16, wherein the at least one amino acid is N-acetyl-L-alanine and the N-acetyl-L-alanine has a concentration in the ophthalmic pharmaceutical composition of between about 0.25 mass % to about 1.5 mass %.

Embodiment 24: The ophthalmic pharmaceutical composition of embodiment 14, wherein the N-acetyl-L-alanine has a concentration of about 0.5 mass %.

Embodiment 25: The pharmaceutical composition of embodiment 1, wherein the ophthalmic pharmaceutical composition is stable for at least 30 days when refrigerated.

Embodiment 26: The pharmaceutical composition of embodiment 25, wherein the ophthalmic pharmaceutical composition is stable for at least 60 days when refrigerated.

Embodiment 27: The pharmaceutical composition of embodiment 26, wherein the ophthalmic pharmaceutical composition is stable for at least 90 days when refrigerated.

Embodiment 28: The pharmaceutical composition of embodiment 27, wherein the ophthalmic pharmaceutical composition is stable for at least 120 days when refrigerated.

Embodiment 29: The pharmaceutical composition of embodiment 28, wherein the ophthalmic pharmaceutical composition is stable for at least 150 days when refrigerated.

Embodiment 30: The pharmaceutical composition of embodiment 29, wherein the ophthalmic pharmaceutical composition is stable for at least 180 days when refrigerated.

Embodiment 31: The pharmaceutical composition of embodiment 30, wherein the ophthalmic pharmaceutical composition is stable for at least 365 days when refrigerated.

Embodiment 32: The pharmaceutical composition of embodiment 1, wherein the ophthalmic pharmaceutical composition is potent for at least 30 days when refrigerated.

Embodiment 33: The pharmaceutical composition of embodiment 32, wherein the ophthalmic pharmaceutical composition is potent for at least 60 days when refrigerated.

Embodiment 34: The pharmaceutical composition of embodiment 33, wherein the ophthalmic pharmaceutical composition is potent for at least 90 days when refrigerated.

Embodiment 35: The pharmaceutical composition of embodiment 34, wherein the ophthalmic pharmaceutical composition is potent for at least 120 days when refrigerated.

Embodiment 36: The pharmaceutical composition of embodiment 35, wherein the ophthalmic pharmaceutical composition is potent for at least 150 days when refrigerated.

Embodiment 37: The pharmaceutical composition of embodiment 36, wherein the ophthalmic pharmaceutical composition is potent for at least 180 days when refrigerated.

Embodiment 38: The pharmaceutical composition of embodiment 37, wherein the ophthalmic pharmaceutical composition is potent for at least 365 days when refrigerated.

Embodiment 39: A method for treating an ophthalmological disease, condition, disorder, syndrome or pathology in a mammalian subject in need of such treatment, the method comprising administering to a patient in need thereof a pharmaceutically effective quantity of an ophthalmic pharmaceutical composition of claim 1.

Embodiment 40: The method of embodiment 39, wherein the ophthalmological disease, condition, disorder, syndrome or pathology is selected from the group consisting of microbial keratitis, endophthalmitis prophylaxis post cataract surgery, and infection prophylaxis for corneal prosthetic implant.

Embodiment 41: The method of embodiment 40, wherein the ophthalmic pharmaceutical composition is administered topically.

Embodiment 42: The method of embodiment 41, wherein the ophthalmic pharmaceutical composition is administered via eyedrops.

### EXAMPLES

The following examples are provided to further elucidate the advantages and features of the present application but are not intended to limit the scope of the application. The examples are for the illustrative purposes only. USP pharmaceutical grade products were used in preparing the formulations described below.

### Example 1. Preparing a Pharmaceutical Composition

A pharmaceutical composition was prepared as described below. The following products were used in the amounts and concentrations specified:
(1) about 2.5 g of vancomycin HCl powder;
(2) about 1.5 g of tobramycin sulfate powder;
(3) about 1.0 g of lidocaine HCl powder;
(4) a quantity of hydrochloric acid for pH adjustment; and
(5) about 100 ml of sterile water for injection, quantum sufficiat.

Vancomycin HCl, tobramycin sulfate and lidocaine HCl powders were combined with a quantity of sterile water for injection to obtain about 90% of the required volume. The so combined products were then mixed until completely dissolved and a clear solution was obtained. The pH of the solution was adjusted to about 5.5±0.1 using hydrochloric acid with mixing, followed by addition of the rest of water.

The solution obtained as described above was filtered through a 0.22-micron filter into sterile 11 mL Nemera vials, and capped with PF10 caps.

The final product was tested chromatographically (HPLC) for potency and stability after being stored for 6 months. The results are shown in Table 1, below. For the comparison, the potency and stability were also measured for tobramycin alone (Table 2), vancomycin alone (Table 3) and a tobramycin-vancomycin system (Table 3). As the results presented in Tables 1-4 below demonstrate, the composition of the present invention (Table 1) remained stable and retained its potency. No visually perceptible change of color was observed. However, only the concentration of tobramycin decreased slightly (to 98% and 96%) after 150-180 days of storage. Each of tobramycin and vancomycin alone withstood the storage for 180 days very well (Tables 2 and 3). According to the USP rules, at least a 90% concentration is required for the product to be acceptable.

However, when the system without lidocaine is compared with that containing lidocaine, it can be sees that the concentration of vancomycin falls to about 91% and below after 120 days storage for the system having no lidocaine (Table 4). At the same time, the concentration of vancomycin remained high (at least 108% for the same storage times) when lidocaine is present in the composition (Table 1). That is a significant and unexpected improvement that may be attributed to the stabilizing effect of lidocaine.

TABLE 1
The Stability Results for the Vancomycin-Tobramycin-Lidocaine System
(composition as in Example 1, stored at −4° C. in a dark place)

| Day | Vancomycin, % | Tobramycin, % | Lidocaine, % |
|---|---|---|---|
| 0 | 112 | 115 | 100 |
| 30 | 112 | 109 | 101 |
| 60 | 112 | 102 | 101 |
| 90 | 107 | 102 | 101 |
| 120 | 108 | 100 | 101 |
| 150 | 109 | 98 | 101 |
| 180 | 110 | 96 | 101 |

TABLE 2
The Stability Results for Tobramycin Alone
(1.5% aqueous solution, stored in a dark place at room temperature)

| Day | Tobramycin, % |
|---|---|
| 0 | 117 |
| 30 | 117 |
| 60 | 119 |
| 90 | 119 |
| 120 | 117 |
| 150 | 116 |
| 180 | 114 |

TABLE 3
The Stability Results for Vancomycin Alone
(5.0 % aqueous solution, stored in a dark place at room temperature)

| Day | Vancomycin, % |
|---|---|
| 0 | 113 |
| 30 | 106 |
| 60 | 111 |
| 90 | 106 |
| 120 | 102 |
| 150 | 102 |
| 180 | 102 |

TABLE 4
The Stability Results for Tobramycin-Vancomycin System
(aqueous solution, 1.5 % tobramycin, 5% vancomycin, stored
at −4° C. in a dark place)

| Day | Tobramycin, % | Vancomycin, % |
|---|---|---|
| 0 | 108 | 108 |
| 30 | 107 | 108 |
| 60 | 105 | 106 |
| 90 | 102 | 107 |
| 120 | 103 | 91 |
| 150 | 108 | 83 |
| 180 | 110 | 81 |

Although the invention has been described with reference to the above examples, it will be understood that modifications and variations are encompassed within the spirit and scope of the invention. Accordingly, the invention is limited only by the following claims.

### Example 2. Preparing a Second Pharmaceutical Composition

A pharmaceutical composition of the present disclosure was prepared comprising the following formula:
(1) 5 g vancomycin HCl;
(2) 1.7 g tobramycin sulfate;
(3) 1 g lidocaine hydrochloride monohydrate
(4) 0.1 g sodium metabisulfite
(5) 0.005 g benzalkonium chloride;
(6) 0.9 g L-glycine
(7) 0..625 g sodium chloride;
(8) 0.1 g hypromellose; and
(9) 100 g water for injection.

The composition was then prepared as described in Example 1. The stability of this composition was then monitored for a period of 180 days.

| | Day | Day | Day | Day | Day | USP Assay |
|---|---|---|---|---|---|---|
| | 0 | 30 | 60 | 90 | 180 | Range |
| Vanco- | 104.9% | 104.0% | 99.1% | 99.8% | 100.5% | 90%-115% |
| mycin | | | | | | |
| Tobra- | 102.3% | 106.9% | 98.9% | 97.6% | 97.9% | 90%-120% |
| mycin | | | | | | |
| Lidocaine | 100% | 102.2% | 96.1% | 100.6% | 102.7% | 95%-105% |
| HCl | | | | | | |

### Example 3. Preparing a Third Pharmaceutical Composition

A pharmaceutical composition of the present disclosure was prepared comprising the following formula:
(1) 5 g vancomycin HCl;
(2) 1.7 g tobramycin sulfate;
(3) 0.1 g sodium metabisulfite
(4) 0.005 g benzalkonium chloride;
(5) 0.5 g N-acetyl-D-alanine
(6) 0.625 g sodium chloride;
(7) 0.1 g hypromellose; and
(8) 100 g water for injection.

1. An ophthalmic pharmaceutical composition, comprising:
(a) a therapeutically effective quantity of at least one antibiotic selected from the group consisting of a glycopeptide antibiotic and an aminoglycoside antibiotic;
(b) at least one amino acid; and
(c) a pharmaceutically acceptable aqueous carrier therefor.

2. The ophthalmic pharmaceutical composition of claim 1, wherein the glycopeptide antibiotic is selected from the group consisting of vancomycin, decaplanin, ramoplanin, cefuroxime, teicoplanin, telavancin, corbomycin, complestatin, and any combination thereof.

3. The ophthalmic pharmaceutical composition of claim 2, wherein the glycopeptide antibiotic is vancomycin.

4. The ophthalmic pharmaceutical composition of claim 1, wherein the aminoglycoside antibiotic is selected from the group consisting of tobramycin, kanamycin A, amikacin, dibekacin, gentamicin, sisomicin, netilmicin, neomycin B, neomycin C, paromomycin, plazomicin, streptomycin, and any combination thereof.

5. The ophthalmic pharmaceutical composition of claim 4, wherein the am inoglycoside antibiotic is tobramycin.

6. The ophthalmic pharmaceutical composition of claim 1, further comprising a therapeutically effective quantity of at least one anesthetic compound.

7. The ophthalmic pharmaceutical composition of claim 6, wherein the at least one anesthetic compound is selected from the group consisting of lidocaine, tetracaine, proparacaine, procaine, dyclonine, chloroprocaine, and any combination thereof.

8. The ophthalmic pharmaceutical composition of claim 6, wherein the at least one anesthetic compound has a concentration in the ophthalmic pharmaceutical composition of between about 0.25 mass % and about 5.0 mass %.

9. The ophthalmic pharmaceutical composition of claim 8, wherein the at least one anesthetic compound has a concentration of about 1.0 mass %.

10. The ophthalmic pharmaceutical composition of claim 7, wherein the anesthetic compound is lidocaine.

11. The ophthalmic pharmaceutical composition of claim 1, wherein the concentration of glycopeptide antibiotic(s) is between about 1.0 mass % and about 10.0 mass %.

12. The ophthalmic pharmaceutical composition of claim 1, wherein the concentration of aminoglycoside antibiotic(s) is between about 1.0 mass % and about 5.0 mass %.

13. The ophthalmic pharmaceutical composition of claim 1, wherein the composition has a pH of between about 4.0 and about 6.0.

14. The ophthalmic pharmaceutical composition of claim 13, wherein the composition has a pH of between about 5.1 to about 5.3.

15. The ophthalmic pharmaceutical composition of claim 1, wherein the at least one amino acid is selected from the group consisting of L-glycine, L-cysteine, L-lysine, N-acetyl-D-alanine, and any combination thereof.

16. The ophthalmic pharmaceutical composition of claim 15, wherein the at least one amino acid has a concentration in the pharmaceutical composition between about 0.1 mass % and about 1.5 mass %.

17. The ophthalmic pharmaceutical composition of claim 16, wherein the at least one amino acid is L-glycine and the L-glycine has a concentration in the ophthalmic pharmaceutical composition of between about 0.1 mass % to about 1.0 mass %.

18. (canceled)

19. The ophthalmic pharmaceutical composition of claim 16, wherein the at least one amino acid is L-cysteine and the L-cysteine has a concentration in the ophthalmic pharmaceutical composition of between about 0.1 mass % and about 0.2 mass %.

20. (canceled)

21. The ophthalmic pharmaceutical composition of claim 16, wherein the at least one amino acid is L-lysine and the L-lysine has a concentration in the ophthalmic pharmaceutical composition of between about 0.1 mass % to about 1.0 mass %.

22. (canceled)

23. The ophthalmic pharmaceutical composition of claim 16, wherein the at least one amino acid is N-acetyl-L-alanine and the N-acetyl-L-alanine has a concentration in the ophthalmic pharmaceutical composition of between about 0.25 mass % to about 1.5 mass %.

24. (canceled)

25. The pharmaceutical composition of claim 1, wherein the ophthalmic pharmaceutical composition is stable for at least 30 days when refrigerated.

26-29. (canceled)

30. The pharmaceutical composition of claim 25, wherein the ophthalmic pharmaceutical composition is stable for at least 180 days when refrigerated.

31. (canceled)

32. The pharmaceutical composition of claim 1, wherein the ophthalmic pharmaceutical composition is potent for at least 30 days when refrigerated.

33-38. (canceled)

39. A method for treating an ophthalmological disease, condition, disorder, syndrome or pathology in a mammalian subject in need of such treatment, the method comprising administering to a patient in need thereof a pharmaceutically effective quantity of an ophthalmic pharmaceutical composition of claim 1.

40-42. (canceled)
